(19) United States
(12) Patent Application Publication
(10) Pub. No.: US 2013/0309685 A1
(43) Pub. Date: Nov. 21, 2013

(54) METHOD FOR TARGET BASED CANCER CLASSIFICATION, TREATMENT, AND DRUG DEVELOPMENT

(71) Applicant: Karim Iskander, Houston, TX (US)

(72) Inventor: Karim Iskander, Houston, TX (US)

(21) Appl. No.: 13/494,993

(22) Filed: Jun. 13, 2012

Related U.S. Application Data
(60) Provisional application No. 61/649,003, filed on May 18, 2012.

(51) Int. Cl. C12Q 1/68

(52) U.S. Cl. CPC C12Q 1/6886
USPC 435/6.14

(57) ABSTRACT

A method of classifying a cancerous tumor is described and comprises the steps of: screening a set of targetable events within a tumor, determining a profile for tumor, and classifying the tumor based on the variant profile of the tumor. More specifically, the tumor is defined and classified based on targetable events; histology and disease stage are not considered. The method will result in greater numbers of samples for clinical studies and better, more accurate combinatorial approaches for treatment. This method overcomes the biases of traditional cancer classification schemes, and advances personalized medicine in solid tumor cancers.

### RELATED U.S. APPLICATIONS

The present application claims priority under U.S. Code Section 119(e) from a provisional patent application, U.S. Patent Application No. 61/496,003, filed on 12 Jun. 2011 and entitled “METHOD FOR TARGET BASED CANCER CLASSIFICATION, TREATMENT, DRUG DEVELOPMENT”.

### STATEMENT REGARDING FEDERALLY SPONSORED RESEARCH OR DEVELOPMENT

Not Applicable.

### REFERENCE TO MICROFICHE APPENDIX

Not Applicable.

### BACKGROUND OF THE INVENTION

1. Field of the Invention
The present invention is in the field of solid tumor cancers. More particularly, the invention relates to methods of classifying solid tumors based on the presence of targetable events, validating the resulting classifications, and applying treatment regimens based on classifications of the solid tumors. Methods for determining the profile of targetable events and determining a classification for a cancer are provided.

2. Description of Related Art Including Information Disclosed Under 37 C.F.R. 1.97 and C.F.R. 1.98.

Prior technology in the field of solid cancer tumors relies upon a classification of the cancer based on histology and tissue of origin (e.g., colon cancer, small cell lung cancer, etc.). These histological classifications can then be further refined using the degree, or stage, of differentiation and invasiveness into other tissues (e.g., Stage 1I colon cancer). Treatment regimens are often prescribed using this overly simple classification scheme.

With the elucidation of the human genome, genetic variants contributing to cancer phenotypes have been identified and validated as contributoring elements in cancer etiology. Treatment regimens have been designed, evaluated in clinical studies, and are now prescribed after screening a cancerous tissue sample for the genetic variant of interest. The most successful and well-publicized example of this targeted therapy is the approval of Imantinib (Gleevec) for treatment of Chronic Myeloid Leukemia (CML) in 2001. However, CML is a very unique cancer because it is driven by a single translocation (bcr-abl), and the one-hit/one-cancer type is not a successful approach to designing treatment regimens for more complex cancer genotypes.

Most cancers are driven by multiple genetic variants or mutations and epigenetic changes. With few exceptions, the two-hit hypothesis is an accurate description of cancer etiology. Essentially, the two-hit hypothesis posits that at a minimum two driving events are needed for tumor development. The etiologically important “two hits” are often single nucleotide polymorphisms (SNP) or other genetic variants that may result in an abnormal cellular state and tumor generation. Further accumulated genetic changes drive invasiveness and resistance to anticancer agents.

Many pharmaceuticals are being developed to target variants that contribute to certain cancers, but they are often limited to particular tissue type cancers. “Dirty kinases” that hit several targets show partial success in some cancers, specifically Renal Cell Carcinoma. However, many Renal Cell Carcinoma patients are refractory to these pharmaceutical agents, while other patients have only modest responses such as partial tumor shrinkage or a prolonged stable disease-state or remission that eventually relapses.

Some pharmaceuticals or other treatment regimens are designed specifically for subpopulations of a particular tissue-type cancer. For example, BRAF inhibitors are selectively used in BRAF-positive melanomas because 70-80% of melanomas are BRAF-positive. There is evidence of a lack of BRAF-inhibitor activity in BRAF-positive tumors, presumably due to the concomitant PI3K pathway activation in these tumors. Relatedly, many BRAF-positive melanoma patients do not respond to BRAF inhibitors presumably because of compensatory mechanisms or other mutations in alternative pathways. However, some patients who would benefit from BRAF inhibitor treatment are often excluded from such treatments because based on the histology of the tumor, the patients are excluded from such treatment protocols. For example, BRAF inhibitors are seldom used in colon cancer (5-7% BRAF-positive rate) or other tissue-specific cancers with small incidence rates.

Incremental, slow progress is being made toward better and more specific therapies and personalized medicine (e.g., BRAF and MEK inhibitors in BRAF-positive melanomas and PARP inhibitors in variant BRCA1 breast cancer and ovarian cancer). Unfortunately, advancing treatment regimens are limited by the current cancer classification scheme (i.e., stage/tissue type) and management of the disease. Targeting one out of several driving mutations can only benefit a small subset of patients, resulting mostly in modest responses and clinical benefit, but targeting smaller subsets of cancer patients with combination targeted therapies will yield a population of patients too small for meaningful and decisive clinical studies. For example, targeting melanoma patients with BRAF and PI3K mutations with a combination of BRAF/MEK pathway inhibitor and a PI3K/mTOR pathway inhibitor, will most likely yield a study population size too small to generate the statistically significant results for safety and effectiveness, as required for FDA approval of the treatment regimen.

An alternative approach may be to use the BRAF/MEK pathway inhibitor and a PI3K/mTOR pathway inhibitor cocktail in all melanoma patients as the population size may achieve statistically significant differences between the treatment and placebo populations. The likelihood in such an approach is that only a very small percentage of patients will receive a benefit for the treatment as this “targeted treatment” is not actually being applied in a targeted manner. Rather, a large number of patients will be treated unnecessarily because their cancer will be non-responsive to the treatment. Non-melanoma cancer patients whose tumors are driven mostly by mutations in these two pathways will be completely ignored.

There are many examples of genetic factors contributing to cancer. Microsatellite Instability (MSI) from deficiencies in mismatch DNA repair (MMR) is an initiating factor and a predictive factor in several cancers including colorectal, endometrial, ovarian, and gastric cancers. BRAF mutations are present in 80% of melanomas, 1-3% of lung cancer, and approximately 5% of colorectal cancer. KRAS mutations are implicated in lung adenocarcinoma, ductal carcinoma of the pancreas, and colorectal carcinoma. Thus, common targetable events found in multiple tissue type tumors can lead new combinatorial treatment regimens independent of any histological or disease progression classifications.

The prior art contains methods for classifying cancers, but these methods typically involve a tissue dependent approach. Essentially, the methods described are specialized methods directed towards tumors of specific tissues of origin. U.S. Pat. No. 7,781,179 describes screening for genetic abnormalities that can be causative, disease susceptibility, or drug responsiveness variants or otherwise linked to bladder cancer. The screening for bladder cancer variation is performed in a tissue specific manner, specifically a subpopulation of urothelial basal cells. The inventors hypothesize that these particular larger cells preferentially accumulate genetic and epigenetic variation that is caused by physical or chemical assault.

Prior art methods of characterizing cancers often involve gene expression profiles. Expression profiles are compiled for cancerous tumors and compared to wildtype or noncancerous expression profiles to identify those expression profiles associated with the particular cancer. U.S. Patent Application No. 2012/0064520 also involves bladder cancer and is a method of classification based on gene expression profiles. U.S. Pat. No. 7,943,306 involves detecting core serum response (CSR) profiles. Induced CSR signatures are suggested to indicate a higher probability of metastasis. Classification according to CSR response profiles allows optimization of treatment protocols.

Methods for testing selected compounds against cancerous tumors can also be found in the prior art. U.S. Pat. No. 7,118,853 explains a method for utilizing expression profiles in identified genes and gene subsets that are useful for classifying breast cancer. These genes and gene subsets are probable contributors to breast cancer development, progression, and response to therapy.

A method of characterizing and classifying solid tumor cancers that is independent of tissue type or stage of disease is desired. Such a method will allow researchers to include greater numbers of samples to achieve statistical significance in drug development and clinical trials of treatment regimens. Furthermore, such a method will advance the principle of personalized medicine in that a patient's cancer will be characterized based on targetable events, and presence of targetable events will result in tailored therapies for the individual.

### SUMMARY OF THE INVENTION

The present invention relates to the classification of cancers based on the presence of genetic and epigenetic predictive events. In particular, the present invention relates to classifying cancers based on profiles of a cancer generated by screening for targetable events that contribute to the cancer with no regard to the tissue of origin or to the particular stage of the disease. The classifications of the present invention are useful for prognostic evaluation of patients; for developing, testing, and validating proposed treatment regimens; and for predicting a patient's responsiveness to treatment regimens.

It is an object of the present invention to provide a method capable of characterizing and classifying a solid cancer tumor, regardless of the tissue of origin of the cancer.

It is a further object of the present invention to provide a method of characterizing and classifying a solid cancer tumor that enables researchers to enhance the sample size in laboratory and clinical trials for statistical validation of associating classifications and treatment regimens.

It is a further object of the invention to provide a method of characterizing and classifying a solid cancer tumor that will fulfill the potential of personalized medicine.

It is a further object of the invention to provide a method of characterizing and classifying a solid cancer tumor that is applicable in defining what treatment regimen to use and matching the patient with the right combination of targeted therapies.

It is a further purpose of the invention to provide a method of characterizing and classifying a solid cancer tumor that provides a new and applicable path of developing cancer therapies across all tumor histologies based on the genetic make-up of the tumor.

### BRIEF DESCRIPTION OF THE DRAWINGS

FIG. 1 is a diagram of one embodiment of the method.

### DETAILED DESCRIPTION OF THE INVENTION

A method of classifying a cancerous tumor is described and comprises the steps of: screening a set of targetable events within a tumor, determining a profile for tumor, and classifying the tumor based on the variant profile of the tumor. A tumor classification in the present invention consists of a profile is defined by at least two targetable events. In general, targetable events will be a suspected direct or indirect contributor to a solid tumor cancer and can be detected by screening for the targetable events either directly or indirectly.

The present invention is based on the realization that the current approach to defining cancers is myopic and rigid. Defining a cancer type based on tissue type gives researchers little incentive to discover common underlying events that cancers possess, even in different tissue types. Defining a cancer by factors other than tissue type, and therefore not constrained histologically, will allow researchers to increase the number of samples studied for statistical purposes.

The first step in the method of classifying a solid cancer tumor is to identify genes that may contribute to the disease state. The disease state can be any stage of cancer progression. Contributing to a disease state may refer to a causative event, a modest modifier of the disease phenotype, or any other event that can potentially affect the disease. This compilation is usually accomplished by thoroughly reviewing the literature and identifying those genes, genetic variants, epigenetic modifications, and other potentially causative contributors. While this “candidate” approach may not include every possible contributor, it will eliminate much of the noise seen in whole genome approaches where thousands of potential contributors are assayed.

TABLE 1 is a list of genes that may harbor potential targetable events that contribute to solid cancer tumors. Each gene in the list has been correlated with cancer in previous studies. While this list is a preferred set of genes to screen for targetable events that potentially contribute to solid cancer tumors, it is not an exhaustive list. Screening these genes for targetable events tissues taken from solid tumors, regardless of tissue or stage classification, will increase the probability of finding statistically significant profiles for further study. Furthermore, some genetic variation occurs at the epigenetic level (e.g., methylation) and can be included in the list of contributors that will be screened. As technological advances improve the sensitivity and reliability of high-throughput assays such as microarrays, these genome-wide assays may be utilized in lieu of the candidate approach.

Anaplastic Lymphoma Kinase (ALK) is included in the list of genes to be screened because it has been validated by the development of crizotinib for ALK+ non-small cell lung cancer lung cancer.

B-Cell CLL/Lymphoma 2 (Bcl-2) is included in the list of genes to be screened because it has been validated in phase I and phase II clinical studies of obatoclax in small cell lung cancer.

(BRAF) is included in the list of genes to be screened because it has been validated by the clinical studies and development of vemurafenib in BRAF mutation positive melanoma.

Breast Cancer 1 and 2 Gene (BRCA1 and BRCA2) are included in the list of genes to be screened because they have been validated in several phase II studies to predict response to PARP inhibitors (olaparib, veliparib, iniparib) in breast and ovarian cancer.

v-Kit Hardy-Zuckerman 4 Feline Sarcoma Viral Oncogene (Kit) is included in the list of genes to be screed because it has been validated as a driver for some tumors like gastrointestinal stromal tumor (GIST) and tyrosine kinase inhibitors that inhibit Kit demonstrated activity in several phase II studies, and the FDA approved this treatment regiment for patients with GIST.

Met Protooncogene (Met) is included in the list of genes to be screened because Met has been established in preclinical studies as a driver for certain tumor development, invasiveness and metastasis. Phase I studies of Met inhibitors like ARQ 197 demonstrated clinical activity in subgroups of colorectal cancer and lung cancer.

Epidermal Growth Factor Receptor (EGFR) is included in the list of genes to be screened because EGFR expression correlated with response to EGFR inhibitors like Cetuximab in head and neck, colorectal, and lung cancer.

Focal Adhesion Kinase (FAK) is included in the list of genes to be screened because FAK has been recently established as a contributor in cancer progression and inhibitors of FAK like PF-00562271 demonstrated clinical activity in subset of advanced cancer patients.

V-ERB-B2 Avian Erthyroblastic Leukemia Viral Oncogene Homolog 2 (HER-2) is included in the list of genes to be screened because it has been validated to predict response to anti-HER2 antibody trastuzumab and HER2 inhibitor lapatinib.

V-KI-Ras 2 Kirsten Rat Sarcoma Viral Oncogene Homolog (KRAS) is included in the list of genes to be screened because it has been established to predict response to panitumumab in colorectal cancer patients and also established as a contributor in cancer development and is of prognostic value.

FKBP12—Rapamycin Complex-Associated Protein (mTOR) is included in the list of genes to be screened because the PI3K-AKT-mTOR has been well established as a pathway for tumorigenesis and mTOR inhibition demonstrated clinical activity in several tumors and is approved for renal cell carcinoma.

Phosphatidylinositol 3-Kinase, Catalytic, Alpha (PI3KCA) is included in the list of genes to be screened because as the PI3K-AKT-mTOR has been well established as a pathway for tumorigenesis and recent clinical data demonstrated promising activity for PI3K inhibitors and correlation with PI3KCA mutations.

Rearranged During Transfection Protooncogene (RET) is included in the list of genes to be screened because activating mutations in RET are associated with cancer development specially thyroid cancer and various endocrine cancer. Recently, RET inhibitors like XL-184 and vandetanib demonstrated activity in tumors with high incidence of RET mutation, and vandetanib was recently approved as a pharmaceutical treatment for medullary thyroid cancer.

Vascular Endothelial Growth Factor A (VEGF) is included in the list of genes to be, screened because anti VEGF (Bevacizumab) and anti-VEGFR (Sorafenib, sunitinib, Tivozanib) demonstrated activity in tumors known to have high levels of VEGF and VEGFR.

Additional genes that may harbor targetable events are abundant and can be included in the screening process. Additional genes may be studied pre-clinically, in tumor samples, or otherwise followed to assess the effectiveness of targeting these additional events with small molecules or biological to evaluate their possible addition to the preferred fifteen targetable events.

Table 2 is a list of additional genes that may harbor targetable events that may play an etiological role in solid tumor cancer. One skilled in the art would recognize that the list of genes that harbor targetable events that contribute to cancer expands well beyond this list and that this list is a preferred, but not exhaustive, list of genes to be screened. Each of the genes listed has been linked to cancer in previous studies, but additional targetable events need not be just genes or variants therein. Epigenetic modifications, translocations, insertions, deletions as well as environmental inputs (e.g., carcinogen exposure) can be targetable events as well.

Signal Transducer and Activator of Transposition 3 (STAT3) is included in, the list of additional targetable events because it has been established player in tumorigenesis and several inhibitors are now in preclinical and early clinical investigation.

Fibroblast Activation Protein, Alpha (FAP) is included in the list of additional targetable events because it has been identified as a substantial contributor to tumor progression and metastasis and several targeting modalities are under investigation.

Fibroblast Growth Factor Receptors 1-4 (EGFR 1-4) are included in the list of additional targetable events because they have been implicated in breast, hepatic and lung cancer and inhibitors of FGFRs are in preclinical and early clinical development.

PIM Oncogene (PIM) is included in the list of additional targetable events because it has been discovered to play a prominent role in development of sarcoma and metastasis. PIM inhibitor studies are ongoing.

Insulin-like Growth Factor 1 Receptor (IGF1R) is included in the list of additional targetable events because it has been implicated in cancer development and phase I/II studies of targeting inhibitors are enrolling patients.

Neuroblastoma Ras Viral Oncogene Homolog (NRAS) is included in the list of additional targetable events because preclinical data shows possible predicative value for NRAS mutation in regards to inhibitors of downstream MEK. Clinical studies with molecular screening for NRAS, MEK and BRAF mutations are ongoing.

A set of genes will be screened for targetable events to determine a profile for a sample. A sample can be material obtained in a biopsy, a tissue bank or other repository, a blood draw, or any other material that may be used to generate useful information concerning targetable events or cancerous or normal states. The material can be in any form including genetic material, tissue samples, proteins, or any other material that may be used to generate useful information regarding targetable events or cancerous or normal states. While screening is a required step for the method, no particular screening method is required. For instance, detecting genetic variation in a gene can be accomplished by sequencing the gene but particular single nucleotide polymorphisms (SNPs) can be screened for directly using microarray analysisor other commercially available or proprietary methods. In some embodiments of the invention, genes are screened for targetable events, but in alternative embodiments, known targetable events are screened for directly in samples. In one embodiment of the invention, screening a set of genes for targetable events will consist of amplifying the exonic, and adjacent, regions of the genes by polymerase chain reaction (PCR) or other amplification means. The amplified regions of interest will then be used as templates in sequencing reactions to determine the sequence of the regions of interest. Known genetic variants can be detected while unknown variants, such as rare variants that have not been discussed in the literature, can be detected by comparing the sample's sequence to a wildtype, or reference, sequence.

In another embodiment of the invention, the regions of interest will not be sequenced, but rather, known genetic variation such as deletions, insertions, single nucleotide polymorphisms (SNPs), and rare variants will be screened directly.

Many of the embodiments described above utilize nucleotide resolution detection methods for detecting genetic variation, one skilled in the art will understand that the methods used to screen for targetable events can result in nucleotide resolution, but lower resolution methods, as well as non-genetic methods, can be used as well. For example, in one embodiment, translocations can be screened for using karyotype analysis. Furthermore, the material used for screening can be any material which can be used to characterize a tumor. For instance, deoxyribonucleic acid isolated from a tumor biopsy sample could be used to screen for targetable events such as genetic variants. Isolated ribonucleic acid (RNA) could be used to determine an expression profile that could aid in classifying a tumor. Also, whole blood samples could be used to screen for targetable events such as aberrant protein levels caused by a tumor.

In another embodiment of the invention, the targetable events screened for may include epigenetic variation such as methylation. There are numerous categories of epigenetic variation and one skilled in the art would recognize the invention is not limited to any particular type of epigenetic variation to provide the data necessary to classify a cancerous tumor.

Results of screening for targetable events are used to assemble a profile for the sample. A profile can consist of the entire screening results or a subset of the results. A preferred profile would consist of each gene screened being characterized as positive or negative for targetable events. For example, if FAP, Bcl-2, and ALK are screened, and three SNPs are detected in FAP, a deletion is detected in BLC-2, and no targetable events are detected in ALK, the profile of the three screened genes could be FAP+/Bcl-2+/ALK. Alternative profile reporting is available, such as including in the profile only those genes screened that contain targetable events. Using such a profile reporting scheme for the example above would result in the following profile: FAP/Bcl-2. One skilled in the art will recognize that a profile can take any number of forms so long as it is descriptive of the samples screened. Individual targetable events, such as a known disease-associated SNP, can also be included in the profile. Including such information can aid in discerning a proper treatment course for a patient or designing a proper clinical trial.

Once a profile has been assembled for a sample, classifications can be assigned. A classification will consist of at least two targetable events. The incidence of each profile can be determined prior to assigning classifications, and in such an embodiment, a cut-off incidence rate would be established and only those profiles with an incidence rater greater than the cut-off incidence rate would be assigned a classification. This would be an efficient means of identifying only those profiles that would allow researchers to conduct statistically significant clinical studies. Lower incidence rate profiles would not yield statistically significant results, and any proposed treatment regimen could not be validated due to low statistical power. Alternatively, every profile can be assigned a classification, and then the incidence of the classification can be determined.

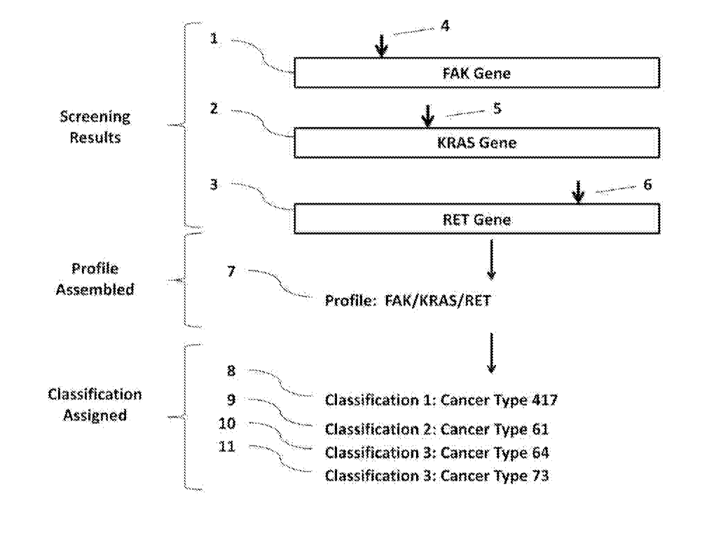

Table 3 is a partial list of classifications based on the detection of targetable events in the gene set listed in Table 1. Table 3 illustrates that a single profile may have multiple classifications. FIG. 1 illustrates the method described herein. The sample screened for the preferred set of genes in Table 1 has a targetable event 4 in the FAK gene 1, a targetable event 5 in the KRAS gene 2, and a targetable event 6 in the RET gene 3. The resulting profile 7 may be written as FAK/KRAS/RET to indicate that targetable events were detected in these three genes. Based on this profile 7, the tumor classification 8 will be Cancer Type 417. The same sample can also be classified as Cancer Type 61 (targetable events detected in FAK and KRAS), Cancer Type 64 (targetable events detected in FAK and RET), and Cancer Type 73 (targetable events detected in KRAS and RET).

As the frequency of any given targetable event is less than 1.0, each additional targetable event will cause the frequency of the profile (Cancer Type) to decrease (with the exception of complete linkage of targetable events, in which case the frequency would remain the same). As the frequency decreases, greater numbers of samples will be required to reach statistical significance. Assigning multiple classifications can allow a researcher to identify those classifications that have a sufficient number of samples to achieve statistical significance.

There are approximately ten million patients afflicted with some form of solid cancer tumor. If the frequency, or prevalence, of one of the Cancer Types listed in Table 3 is 1 in 1000, then there would be approximately ten thousand patients with that particular Cancer Type. This is a large enough number of patients to develop a treatment modality. It is expected that all Cancer Types would meet the Orphan disease status based on the number of patients (i.e., <200,000 patients).

In one embodiment of the invention, an individual patient's tumor sample will be screened for diagnostic and therapeutic purposes. The classification of the tumor will aid the caregiver in determining the proper therapeutic approach. A combination of pharmaceuticals may likely be prescribed because the tumor will have at least two targetable events. In a clinical setting, determination of the incidence rate may not be necessary. An individual patient's profile could be immediately assigned a classification and a treatment regimen assigned based on the profile.

TABLE 1

| NCBI Accession No. | Event | Description |
|---|---|---|
| NG_009445.1 | ALK | Anaplastic Lymphoma Kinas mutations e.g., EML4-ALK |
| NG_009361.1 | Bcl-2 | B-cell lymphoma 2 family including BCL-2 and BCLXL over |
| | | expression and BAX mutation |
| NG_007873.2 | BRAF | Proto-oncogene B-Raf activating mutation; e.g., V600E; other |
| | | mutations include: R461I, I462S, G463E, G463V, G465A, |
| | | G465E, G465V, G468A, G468E, N580S, E585K, D593V, F594L, |
| | | G595R, L596V, T598I, V599D, V599E, V599K, V599R, K600E, |
| | | A727V |
| NG_005905.2 | BRCA | Inactivating mutations in tumor suppressor Breast Cancer |
| (BRCA1) | | Gene 1 (BRCA1) or 2 (BRCA2); e.g., Frameshift mutations |
| NG_012772.3 | | that prevent translation of functional protein |
| (BRCA2) | | |
| NG_007456.1 | cKit | Activating mutations in Mast/stem cell growth factor |
| | | receptor (SCFR), also known as proto-oncogene c-Kit or |
| | | tyrosine-protein kinase Kit or CD117; e.g., activating |
| | | mutations in exon 17 |
| NG_008996.1 | cMet | Overexpression of Proto-oncogene that encodes a protein |
| | | known as hepatocyte growth factor receptor (HGFR), also |
| | | known as MET |
| NG_007726.2 | EGFR | Overexpression or activating mutation in Epidermal Growth |
| | | Factor Receptor; e.g., EGFRvIII mutation, EGFR upregulation |
| NG_029467.1 | FAK | Overexpression of Focal Adhesion Kinase (FAK) |
| NG_007503.1 | HER2 | Amplification/over-expression of HER2: Human Epidermal |
| | | Growth Factor Receptor 2, also known as Neu, ErbB-2, |
| | | CD340 or p185 |
| NG_007524.1 | KRAS | Activating mutations in Kirsten rat sarcoma viral oncogene |
| | | homolog or KRAS; e.g., Activating KRAS mutations include |
| | | codons 12, 13, 59, 61 |
| NM_004958 | mTOR | Loss of PTEN (negative regulator of mTOR), activating |
| | | mutations in AKT1, activating mutations in mTOR, |
| | | hyperphosphorylation of S6K and S6 |
| NG_012113.2 | PI3K | Activating mutations in p110α (PIK3CA) exons 9 and 20 |
| | | [codons 532-554 of exon 9 (helical domain) and |
| | | codons1011-1062 of exon 20 (kinase domain)], or amplified |
| | | PIK3CA |
| NG_007489.1 | RET | Chromosomal rearrangements resulting in Oncoptotein |
| | | RET/PTC or point mutations activating RET like M918T |
| NG_008732.1 | VEGF | Overexpression of VEGF, VEGFR-1, or VEGFR-2 |

TABLE 2

| NCBI Accession No. | Event | Description |
|---|---|---|
| NG_007370.1 | STAT3 | Signal transducer and activator |
| | | of transcription 3 |
| NG_027991.1 | FAP | Fibroblast activation, protein |
| NG_007729.1 | FGFR | Fibroblast growth factor receptors |
| (FGFR1) | | (1-4) |
| NG_012449.1 | | |
| (FGFR2) | | |
| NG_012632.1 | | |
| (FGFR3) | | |
| NG_012067.1 | | |
| (FGFR4) | | |
| NG_029601.1 | PIM | PIM oncogenes 1-3; serine/threonine |
| (PIM1) | | protein kinases of the Pim (proviral |
| NG_016262.1 | | integration of Moloney virus) |
| (PIM2) | | |
| NM_001001852 | | |
| (PIM3) | | |
| NG_009492.1 | IGF-1R | Insulin-like growth factor type I |
| | | receptor e.g., IR-A fetal splice variant |
| NG_007572.1 | NRAS | Neuroblastoma RAS |

TABLE 3

| | | | | |
|---|---|---|---|---|
| Cancer Type | Event 1 | Event 2 | Event 3 | Event 4 |

| | | | |
|---|---|---|---|
| 1 | ALK | Bcl-2 | |
| 2 | ALK | BRAF | |
| 3 | ALK | BRCA | |
| 4 | ALK | cKit | |
| 5 | ALK | cMet | |
| 6 | ALK | EGFR | |
| 7 | ALK | FAK | |
| 8 | ALK | HER2 | |
| 9 | ALK | KRAS | |
| 10 | ALK | mTOR | |
| 11 | ALK | PI3K | |
| 12 | ALK | RET | |
| 13 | ALK | VEGF | |
| 14 | Bcl-2 | BRAF | |
| 15 | Bcl-2 | BRCA | |
| 16 | Bcl-2 | cKit | |
| 17 | Bcl-2 | cMet | |
| 18 | Bcl-2 | EGFR | |
| 19 | Bcl-2 | FAK | |
| 20 | Bcl-2 | HER2 | |
| 21 | Bcl-2 | KRAS | |
| 22 | Bcl-2 | mTOR | |
| 23 | Bcl-2 | PI3K | |
| 24 | Bcl-2 | RET | |
| 25 | Bcl-2 | VEGF | |
| 26 | BRCA | cKit | |
| 27 | BRCA | cMet | |
| 28 | BRCA | EGFR | |
| 29 | BRCA | FAK | |
| 30 | BRCA | HER2 | |
| 31 | BRCA | KRAS | |
| 32 | BRCA | mTOR | |
| 33 | BRCA | PI3K | |
| 34 | BRCA | RET | |
| 35 | BRCA | VEGF | |
| 36 | cKit | cMet | |
| 37 | cKit | EGFR | |
| 38 | cKit | FAK | |
| 39 | cKit | HER2 | |
| 40 | cKit | KRAS | |
| 41 | cKit | mTOR | |
| 42 | cKit | PI3K | |
| 43 | cKit | RET | |
| 44 | cKit | VEGF | |
| 45 | cMet | EGFR | |
| 46 | cMet | FAK | |
| 47 | cMet | HER2 | |
| 48 | cMet | KRAS | |
| 49 | cMet | mTOR | |
| 50 | cMet | PI3K | |
| 51 | cMet | RET | |
| 52 | cMet | VEGF | |
| 53 | EGFR | FAK | |
| 54 | EGFR | HER2 | |
| 55 | EGFR | KRAS | |
| 56 | EGFR | mTOR | |
| 57 | EGFR | PI3K | |
| 58 | EGFR | RET | |
| 59 | EGFR | VEGF | |
| 60 | FAK | HER2 | |
| 61 | FAK | KRAS | |
| 62 | FAK | mTOR | |
| 63 | FAK | PI3K | |
| 64 | FAK | RET | |
| 65 | FAK | VEGF | |
| 66 | HER2 | KRAS | |
| 67 | HER2 | mTOR | |
| 68 | HER2 | PI3K | |
| 69 | HER2 | RET | |
| 70 | HER2 | VEGF | |
| 71 | KRAS | mTOR | |
| 72 | KRAS | PI3K | |
| 73 | KRAS | RET | |
| 74 | KRAS | VEGF | |
| 75 | mTOR | PI3K | |
| 76 | mTOR | RET | |
| 77 | mTOR | VEGF | |
| 78 | PI3K | RET | |
| 79 | PI3K | VEGF | |
| 80 | RET | VEGF | |
| 81 | ALK | Bcl-2 | BRAF |
| 82 | ALK | Bcl-2 | BRCA |
| 83 | ALK | Bcl-2 | cKit |
| 84 | ALK | Bcl-2 | cMet |
| 85 | ALK | Bcl-2 | EGFR |
| 86 | ALK | Bcl-2 | FAK |
| 87 | ALK | Bcl-2 | HER2 |
| 88 | ALK | Bcl-2 | KRAS |
| 89 | ALK | Bcl-2 | mTOR |
| 90 | ALK | Bcl-2 | PI3K |
| 91 | ALK | Bcl-2 | RET |
| 92 | ALK | Bcl-2 | VEGF |
| 93 | ALK | BRAF | BRCA |
| 94 | ALK | BRAF | cKit |
| 95 | ALK | BRAF | cMet |
| 96 | ALK | BRAF | EGFR |
| 97 | ALK | BRAF | FAK |
| 98 | ALK | BRAF | HER2 |
| 99 | ALK | BRAF | KRAS |
| 100 | ALK | BRAF | mTOR |
| 101 | ALK | BRAF | PI3K |
| 102 | ALK | BRAF | RET |
| 103 | ALK | BRAF | VEGF |
| 104 | ALK | BRCA | cKit |
| 105 | ALK | BRCA | cMet |
| 106 | ALK | BRCA | EGFR |
| 107 | ALK | BRCA | FAK |
| 108 | ALK | BRCA | HER2 |
| 109 | ALK | BRCA | KRAS |
| 110 | ALK | BRCA | mTOR |
| 111 | ALK | BRCA | PI3K |
| 112 | ALK | BRCA | RET |
| 113 | ALK | BRCA | VEGF |
| 114 | ALK | cKit | cMet |
| 115 | ALK | cKit | EGFR |
| 116 | ALK | cKit | FAK |
| 117 | ALK | cKit | HER2 |
| 118 | ALK | cKit | KRAS |
| 119 | ALK | cKit | mTOR |
| 120 | ALK | cKit | PI3K |
| 121 | ALK | cKit | RET |
| 122 | ALK | cKit | VEGF |
| 123 | ALK | cMet | EGFR |
| 124 | ALK | cMet | FAK |
| 125 | ALK | cMet | HER2 |
| 126 | ALK | cMet | KRAS |
| 127 | ALK | cMet | mTOR |
| 128 | ALK | cMet | PI3K |
| 129 | ALK | cMet | RET |
| 130 | ALK | cMet | VEGF |
| 131 | ALK | EGFR | FAK |
| 132 | ALK | EGFR | HER2 |
| 133 | ALK | EGFR | KRAS |
| 134 | ALK | EGFR | mTOR |
| 135 | ALK | EGFR | PI3K |
| 136 | ALK | EGFR | RET |
| 137 | ALK | EGFR | VEGF |
| 138 | ALK | FAK | HER2 |
| 139 | ALK | FAK | KRAS |
| 140 | ALK | FAK | mTOR |
| 141 | ALK | FAK | PI3K |
| 142 | ALK | FAK | RET |
| 143 | ALK | FAK | VEGF |
| 144 | ALK | HER2 | KRAS |
| 145 | ALK | HER2 | mTOR |
| 146 | ALK | HER2 | PI3K |
| 147 | ALK | HER2 | RET |
| 148 | ALK | HER2 | VEGF |
| 149 | ALK | KRAS | mTOR |
| 150 | ALK | KRAS | PI3K |
| 151 | ALK | KRAS | RET |
| 152 | ALK | KRAS | VEGF |
| 153 | ALK | mTOR | PI3K |
| 154 | ALK | mTOR | RET |
| 155 | ALK | mTOR | VEGF |
| 156 | ALK | PI3K | RET |
| 157 | ALK | PI3K | VEGF |
| 158 | ALK | RET | VEGF |
| 159 | Bcl-2 | BRAF | BRCA |
| 160 | Bcl-2 | BRAF | cKit |
| 161 | Bcl-2 | BRAF | cMet |
| 162 | Bcl-2 | BRAF | EGFR |
| 163 | Bcl-2 | BRAF | FAK |
| 164 | Bcl-2 | BRAF | HER2 |
| 165 | Bcl-2 | BRAF | KRAS |
| 166 | Bcl-2 | BRAF | mTOR |
| 167 | Bcl-2 | BRAF | PI3K |
| 168 | Bcl-2 | BRAF | RET |
| 169 | Bcl-2 | BRAF | VEGF |
| 170 | Bcl-2 | BRCA | cKit |
| 171 | Bcl-2 | BRCA | cMet |
| 172 | Bcl-2 | BRCA | EGFR |
| 173 | Bcl-2 | BRCA | FAK |
| 174 | Bcl-2 | BRCA | HER2 |
| 175 | Bcl-2 | BRCA | KRAS |
| 176 | Bcl-2 | BRCA | mTOR |
| 177 | Bcl-2 | BRCA | PI3K |
| 178 | Bcl-2 | BRCA | RET |
| 179 | Bcl-2 | BRCA | VEGF |
| 180 | Bcl-2 | cKit | cMet |
| 181 | Bcl-2 | cKit | EGFR |
| 182 | Bcl-2 | cKit | FAK |
| 183 | Bcl-2 | cKit | HER2 |
| 184 | Bcl-2 | cKit | KRAS |
| 185 | Bcl-2 | cKit | mTOR |
| 186 | Bcl-2 | cKit | PI3K |
| 187 | Bcl-2 | cKit | RET |
| 188 | Bcl-2 | cKit | VEGF |
| 189 | Bcl-2 | cMet | EGFR |
| 190 | Bcl-2 | cMet | FAK |
| 191 | Bcl-2 | cMet | HER2 |
| 192 | Bcl-2 | cMet | KRAS |
| 193 | Bcl-2 | cMet | mTOR |
| 194 | Bcl-2 | cMet | PI3K |
| 195 | Bcl-2 | cMet | RET |
| 196 | Bcl-2 | cMet | VEGF |
| 197 | Bcl-2 | EGFR | FAK |
| 198 | Bcl-2 | EGFR | HER2 |
| 199 | Bcl-2 | EGFR | KRAS |
| 200 | Bcl-2 | EGFR | mTOR |
| 201 | Bcl-2 | EGFR | PI3K |
| 202 | Bcl-2 | EGFR | RET |
| 203 | Bcl-2 | EGFR | VEGF |
| 204 | Bcl-2 | FAK | HER2 |
| 205 | Bcl-2 | FAK | KRAS |
| 206 | Bcl-2 | FAK | mTOR |
| 207 | Bcl-2 | FAK | PI3K |
| 208 | Bcl-2 | FAK | RET |
| 209 | Bcl-2 | FAK | VEGF |
| 210 | Bcl-2 | HER2 | KRAS |
| 211 | Bcl-2 | HER2 | mTOR |
| 212 | Bcl-2 | HER2 | PI3K |
| 213 | Bcl-2 | HER2 | RET |
| 214 | Bcl-2 | HER2 | VEGF |
| 215 | Bcl-2 | KRAS | mTOR |
| 216 | Bcl-2 | KRAS | PI3K |
| 217 | Bcl-2 | KRAS | RET |
| 218 | Bcl-2 | KRAS | VEGF |
| 219 | Bcl-2 | mTOR | PI3K |
| 220 | Bcl-2 | mTOR | RET |
| 221 | Bcl-2 | mTOR | VEGF |
| 222 | Bcl-2 | PI3K | RET |
| 223 | Bcl-2 | PI3K | VEGF |
| 224 | Bcl-2 | RET | VEGF |
| 225 | BRAF | BRCA | cKit |
| 226 | BRAF | BRCA | cMet |
| 227 | BRAF | BRCA | EGFR |
| 228 | BRAF | BRCA | FAK |
| 229 | BRAF | BRCA | HER2 |
| 230 | BRAF | BRCA | KRAS |
| 231 | BRAF | BRCA | mTOR |
| 232 | BRAF | BRCA | PI3K |
| 233 | BRAF | BRCA | RET |
| 234 | BRAF | BRCA | VEGF |
| 235 | BRAF | cKit | cMet |
| 236 | BRAF | cKit | EGFR |
| 237 | BRAF | cKit | FAK |
| 238 | BRAF | cKit | HER2 |
| 239 | BRAF | cKit | KRAS |
| 240 | BRAF | cKit | mTOR |
| 241 | BRAF | cKit | PI3K |
| 242 | BRAF | cKit | RET |
| 243 | BRAF | cKit | VEGF |
| 244 | BRAF | cMet | EGFR |
| 245 | BRAF | cMet | FAK |
| 246 | BRAF | cMet | HER2 |
| 247 | BRAF | cMet | KRAS |
| 248 | BRAF | cMet | mTOR |
| 249 | BRAF | cMet | PI3K |
| 250 | BRAF | cMet | RET |
| 251 | BRAF | cMet | VEGF |
| 252 | BRAF | EGFR | FAK |
| 253 | BRAF | EGFR | HER2 |
| 254 | BRAF | EGFR | KRAS |
| 255 | BRAF | EGFR | mTOR |
| 256 | BRAF | EGFR | PI3K |
| 257 | BRAF | EGFR | RET |
| 258 | BRAF | EGFR | VEGF |
| 259 | BRAF | FAK | HER2 |
| 260 | BRAF | FAK | KRAS |
| 261 | BRAF | FAK | mTOR |
| 262 | BRAF | FAK | PI3K |
| 263 | BRAF | FAK | RET |
| 264 | BRAF | FAK | VEGF |
| 265 | BRAF | HER2 | KRAS |
| 266 | BRAF | HER2 | mTOR |
| 267 | BRAF | HER2 | PI3K |
| 268 | BRAF | HER2 | RET |
| 269 | BRAF | HER2 | VEGF |
| 270 | BRAF | KRAS | mTOR |
| 271 | BRAF | KRAS | PI3K |
| 272 | BRAF | KRAS | RET |
| 273 | BRAF | KRAS | VEGF |
| 274 | BRAF | mTOR | PI3K |
| 275 | BRAF | mTOR | RET |
| 276 | BRAF | mTOR | VEGF |
| 277 | BRAF | PI3K | RET |
| 278 | BRAF | PI3K | VEGF |
| 279 | BRAF | RET | VEGF |
| 280 | BRCA | cKit | cMet |
| 281 | BRCA | cKit | EGFR |
| 282 | BRCA | cKit | FAK |
| 283 | BRCA | cKit | HER2 |
| 284 | BRCA | cKit | KRAS |
| 285 | BRCA | cKit | mTOR |
| 286 | BRCA | cKit | PI3K |
| 287 | BRCA | cKit | RET |
| 288 | BRCA | cKit | VEGF |
| 289 | BRCA | cMet | EGFR |
| 290 | BRCA | cMet | FAK |
| 291 | BRCA | cMet | HER2 |
| 292 | BRCA | cMet | KRAS |
| 293 | BRCA | cMet | mTOR |
| 294 | BRCA | cMet | PI3K |
| 295 | BRCA | cMet | RET |
| 296 | BRCA | cMet | VEGF |
| 297 | BRCA | EGFR | FAK |
| 298 | BRCA | EGFR | HER2 |
| 299 | BRCA | EGFR | KRAS |
| 300 | BRCA | EGFR | mTOR |
| 301 | BRCA | EGFR | PI3K |
| 302 | BRCA | EGFR | RET |
| 303 | BRCA | EGFR | VEGF |
| 304 | BRCA | FAK | HER2 |
| 305 | BRCA | FAK | KRAS |
| 306 | BRCA | FAK | mTOR |
| 307 | BRCA | FAK | PI3K |
| 308 | BRCA | FAK | RET |
| 309 | BRCA | FAK | VEGF |
| 310 | BRCA | HER2 | KRAS |
| 311 | BRCA | HER2 | mTOR |
| 312 | BRCA | HER2 | PI3K |
| 313 | BRCA | HER2 | RET |
| 314 | BRCA | HER2 | VEGF |
| 315 | BRCA | KRAS | mTOR |
| 316 | BRCA | KRAS | PI3K |
| 317 | BRCA | KRAS | RET |
| 318 | BRCA | KRAS | VEGF |
| 319 | BRCA | mTOR | PI3K |
| 320 | BRCA | mTOR | RET |
| 321 | BRCA | mTOR | VEGF |
| 322 | BRCA | PI3K | RET |
| 323 | BRCA | PI3K | VEGF |
| 324 | BRCA | RET | VEGF |
| 325 | cKit | cMet | EGFR |
| 326 | cKit | cMet | FAK |
| 327 | cKit | cMet | HER2 |
| 328 | cKit | cMet | KRAS |
| 329 | cKit | cMet | mTOR |
| 330 | cKit | cMet | PI3K |
| 331 | cKit | cMet | RET |
| 332 | cKit | cMet | VEGF |
| 333 | cKit | EGFR | FAK |
| 334 | cKit | EGFR | HER2 |
| 335 | cKit | EGFR | KRAS |
| 336 | cKit | EGFR | mTOR |
| 337 | cKit | EGFR | PI3K |
| 338 | cKit | EGFR | RET |
| 339 | cKit | EGFR | VEGF |
| 340 | cKit | FAK | HER2 |
| 341 | cKit | FAK | KRAS |
| 342 | cKit | FAK | mTOR |
| 343 | cKit | FAK | PI3K |
| 344 | cKit | FAK | RET |
| 345 | cKit | FAK | VEGF |
| 346 | cKit | HER2 | KRAS |
| 347 | cKit | HER2 | mTOR |
| 348 | cKit | HER2 | PI3K |
| 349 | cKit | HER2 | RET |
| 350 | cKit | HER2 | VEGF |
| 351 | cKit | KRAS | mTOR |
| 352 | cKit | KRAS | PI3K |
| 353 | cKit | KRAS | RET |
| 354 | cKit | KRAS | VEGF |
| 355 | cKit | mTOR | PI3K |
| 356 | cKit | mTOR | RET |
| 357 | cKit | mTOR | VEGF |
| 358 | cKit | PI3K | RET |
| 359 | cKit | PI3K | VEGF |
| 360 | cKit | RET | VEGF |
| 361 | cMet | EGFR | FAK |
| 362 | cMet | EGFR | HER2 |
| 363 | cMet | EGFR | KRAS |
| 364 | cMet | EGFR | mTOR |
| 365 | cMet | EGFR | PI3K |
| 366 | cMet | EGFR | RET |
| 367 | cMet | EGFR | VEGF |
| 368 | cMet | FAK | HER2 |
| 369 | cMet | FAK | KRAS |
| 370 | cMet | FAK | mTOR |
| 371 | cMet | FAK | PI3K |
| 372 | cMet | FAK | RET |
| 373 | cMet | FAK | VEGF |
| 374 | cMet | HER2 | KRAS |
| 375 | cMet | HER2 | mTOR |
| 376 | cMet | HER2 | PI3K |
| 377 | cMet | HER2 | RET |
| 378 | cMet | HER2 | VEGF |
| 379 | cMet | KRAS | mTOR |
| 380 | cMet | KRAS | PI3K |
| 381 | cMet | KRAS | RET |
| 382 | cMet | KRAS | VEGF |
| 383 | cMet | mTOR | PI3K |
| 384 | cMet | mTOR | RET |
| 385 | cMet | mTOR | VEGF |
| 386 | cMet | PI3K | RET |
| 387 | cMet | PI3K | VEGF |
| 388 | cMet | RET | VEGF |
| 389 | EGFR | FAK | HER2 |
| 390 | EGFR | FAK | KRAS |
| 391 | EGFR | FAK | mTOR |
| 392 | EGFR | FAK | PI3K |
| 393 | EGFR | FAK | RET |
| 394 | EGFR | FAK | VEGF |
| 395 | EGFR | HER2 | KRAS |
| 396 | EGFR | HER2 | mTOR |
| 397 | EGFR | HER2 | PI3K |
| 398 | EGFR | HER2 | RET |
| 399 | EGFR | HER2 | VEGF |
| 400 | EGFR | KRAS | mTOR |
| 401 | EGFR | KRAS | PI3K |
| 402 | EGFR | KRAS | RET |
| 403 | EGFR | KRAS | VEGF |
| 404 | EGFR | mTOR | PI3K |
| 405 | EGFR | mTOR | RET |
| 406 | EGFR | mTOR | VEGF |
| 407 | EGFR | PI3K | RET |
| 408 | EGFR | PI3K | VEGF |
| 409 | EGFR | RET | VEGF |
| 410 | FAK | HER2 | KRAS |
| 411 | FAK | HER2 | mTOR |
| 412 | FAK | HER2 | PI3K |
| 413 | FAK | HER2 | RET |
| 414 | FAK | HER2 | VEGF |
| 415 | FAK | KRAS | mTOR |
| 416 | FAK | KRAS | PI3K |
| 417 | FAK | KRAS | RET |
| 418 | FAK | KRAS | VEGF |
| 419 | FAK | mTOR | PI3K |
| 420 | FAK | mTOR | RET |
| 421 | FAK | mTOR | VEGF |
| 422 | FAK | PI3K | RET |
| 423 | FAK | PI3K | VEGF |
| 424 | FAK | RET | VEGF |
| 425 | HER2 | KRAS | mTOR |
| 426 | HER2 | KRAS | PI3K |
| 427 | HER2 | KRAS | RET |
| 428 | HER2 | KRAS | VEGF |
| 429 | HER2 | mTOR | PI3K |
| 430 | HER2 | mTOR | RET |
| 431 | HER2 | mTOR | VEGF |
| 432 | HER2 | PI3K | RET |
| 433 | HER2 | PI3K | VEGF |
| 434 | HER2 | RET | VEGF |
| 435 | KRAS | mTOR | PI3K |
| 436 | KRAS | mTOR | RET |
| 437 | KRAS | mTOR | VEGF |
| 438 | KRAS | PI3K | RET |
| 439 | KRAS | PI3K | VEGF |
| 440 | KRAS | RET | VEGF |
| 441 | mTOR | PI3K | RET |
| 442 | mTOR | PI3K | VEGF |
| 443 | mTOR | RET | VEGF |
| 444 | PI3K | RET | VEGF |

Classifications with 4 events may be added based on prevalence of 1 in 1000 or higher

I claim:

1. A method for classifying a solid cancer tumor, said method comprising the steps of:
screening a set of genes in a solid tumor for targetable events;
determining a profile for the targetable events present in the solid tumor; and
assigning a classification to the solid tumor based on the profile of the targetable events.

2. The method of claim 1, wherein the classification is based on a profile comprised of at least two targetable events present in the set of genes screened.

3. The method of claim 1, wherein the solid cancer tumor can be from any tissue type and any stage of progression.

4. The method of claim 1 further compromising a step of determining the incidence of each cancer classification.

5. A method for classifying a solid tumor cancer, said method comprising the steps of:
screening the genes listed in Table 1 in a solid tumor cancer for targetable events;
determining a profile for the set of targetable events detected in the solid tumor; and
assigning a classification to the tumor based on the profile of the targetable events.

6. The method of claim 5, wherein the classification of the tumor is based on at least two targetable events present in the set of genes screened.

7. The method of claim 5, wherein the classification is based on a profile comprised of at least two targetable events.

8. The method of claim 5, wherein the solid cancer tumor can be from any tissue type and any stage of progression.

9. The method of claim 5 further compromising a step of determining the incidence of each cancer classification.

10. A method for classifying a solid tumor cancer, said method comprising the steps of:
screening the genes listed in Table 1 and Table 2 in a solid tumor cancer for targetable events;
determining a profile for the set of targetable events detected in the solid tumor; and
assigning a classification to the tumor based on the profile of the targetable events.

11. The method of claim 10, wherein the classification of the tumor is based on at least two targetable events present in the set of genes screened.

12. The method of claim 10, wherein the classification is based on a profile comprised of at least two targetable events.

13. The method of claim 10, wherein the solid cancer tumor can be from any tissue type and any stage of progression.

14. The method of claim 10 further compromising a step of determining the incidence of each cancer classification.
